(19) United States
(12) Patent Application Publication
(10) Pub. No.: US 2013/0166028 A1
(43) Pub. Date: Jun. 27, 2013

(54) COMPOSITE LAYER INTERVERTEBRAL RETRIEVING DEVICE

(71) Applicants: Chin-Wu SHIEH, Taoyuan City (TW); Sheng-Fa Hsu, New Taipei City (TW); Chien-Ming Lee, Keelung City (TW)

(72) Inventors: Chin-Wu SHIEH, Taoyuan City (TW); Sheng-Fa Hsu, New Taipei City (TW); Chien-Ming Lee, Keelung City (TW)

(73) Assignee: A-Spine Asia Co., Ltd., Taipei City (TW)

(21) Appl. No.: 13/469,475

(22) Filed: May 11, 2012

(30) Foreign Application Priority Data
Dec. 22, 2011 (TW) 100148207

(51) Int. Cl. A61F 2/44

(52) U.S. Cl. 623/17.16

(57) ABSTRACT

A composite layer intervertebral retrieving device implantable between two vertebrae is provided, including a main carrier having a middle layer, and a securing groove on respective surfaces; multiple securing pieces stably coupled to the securing grooves; wherein the middle layer is a macromolecular material having an elastic modulus of between 2 to 22 GPa, close to the original elastic modulus of the spine of between 12 to 18 GPa. The securing pieces are affinitive to and easily fused with osteocytes, and capable of medically imaging. The securing pieces are coupled to the securing grooves, and when the device is implanted between vertebrae, the securing pieces come into direct contact with the osteocytes of the neighboring vertebrae, with securing bulges thereof pressing against the osteocyes, and the middle layer has an elastic modulus similar to that of the vertebrae to prevent the stress shielding effect.

### FIELD OF THE INVENTION

The present invention relates to a composite layer intervertebral retrieving device, more particularly to a device implantable between two neighboring human vertebrae and having a main carrier and multiple securing pieces; said main carrier has an elastic modulus similar to that of the vertebrae, and said securing pieces are affinitive to and easily fused with osteocytes, and are capable of being medically imaged; said device is in direct contact with the osteocytes of the vertebrae.

### BACKGROUND OF THE INVENTION

Conventionally, spinal disorders include injuries resulted from bone fractures, but also predominantly diseases or degeneration of the intervertebral discs; such that the intervertebral spaces are reduced to cause the neural foramen (intervertebral foramen) between the intervertebral joints to become narrowed. Consequently, the spinal nerve roots are compressed, and this leads to numbness or pain in the afflicted patients.

The general treatments available for treating the above-mentioned symptoms mostly involve the surgical implantation of pedicle screws and rods, so as to enlarge the intervertebral space thereof.

An alternative is to surgically implant an artificial intervertebral disc filler (such as a disc cage or a spacer) between two vertebrae, such that the two neighboring vertebrae can be pushed apart to widen the neural foramen therebetween. However, the aforesaid methods do not take the material properties of the implanted devices into consideration, such as the stress from an intervertebral device that is tolerable by normal human spinal tissues, the original elastic modulus of the vertebrae, the association and fusibility between an implanted material and the osteocytes, as well as the following external non-invasive medical imaging used to track the progress after the implantation. Therefore, the general intervertebral fillers are still plagued with many potential issues.

The key to resolve the above issues lies in the induction of osteocyte growth of the vertebrae, so that the osteocytes are allowed to grow and fuse with osteocyte-associative materials, and in the ability of an implant to be made visible under ex vivo medical light sources; such as X-ray imaging, as well as in making the elastic modulus of the implant to be as close to that of the original vertebra as possible, so as to prevent damage to the vertebrae from the stress shielding effect. Therefore, a composite layer intervertebral retrieving device was proposed to address the aforesaid issues.

### SUMMARY OF THE INVENTION

A simple solution to the aforesaid issues is a composite layer intervertebral retrieving device being implantable between two vertebrae, comprising:

a main carrier having a middle layer, and a securing groove provided on respective surfaces thereof;

multiple securing pieces stably coupled to said securing grooves;

characterized in that: said middle layer is a macromolecular material having an elastic modulus of between 2 to 22 GPa, which is close to the original elastic modulus of the vertebrae of between 12 to 18 GPa; said securing pieces are affinitive to and easily fused with osteocytes, and are capable of being imaged by medical penetrating light sources;

each of said securing pieces has a surface securely coupled to the securing groove and has securing bulges disposed on another surface thereof, and when the device is implanted between the vertebrae, the securing pieces come into direct contact with the osteocytes of the neighboring vertebrae, with the securing bulges pressing against said osteocytes, such that the osteocytes are allowed to be fused therewith; said middle layer has an elastic modulus close to that of the vertebrae to prevent further damage to the vertebrae from the stress shielding effect.

It is therefore an object of the invention to provide a composite layer intervertebral retrieving device.

Another object of the invention is to provide a composite layer intervertebral retrieving device being implantable between two neighboring vertebrae, and includes a main carrier being coupled to multiple securing pieces; said main carrier is a medical macromolecular material that has an elastic modulus of between 2 to 22 GPa, and is close to the original elastic modulus of the vertebrae of between 12 to 18 GPa, and said securing pieces are affinitive to and easily fused with osteocytes, and are capable of being medically imaged.

Yet another object of the invention is to provide a composite layer intervertebral retrieving device having said securing pieces being securely coupled to securing grooves of said main carrier, and with securing bulges disposed on another surface thereof to reinforce coupling; when the device is implanted between the vertebrae, the securing pieces come into contact with the osteocytes of the vertebrae, with the securing bulges pressing against said osteocytes, such that the osteocytes are allowed to be fused therewith; the middle layer of said main cattier has an elastic modulus close to that of the vertebrae to prevent further damage to the vertebrae from the stress shielding effect.

A further object of the invention is to provide a composite layer intervertebral retrieving device having said main carrier being made of medical polyetheretherketone (PEEK), and said securing pieces being fabricated from medical titanium alloys or tantalum metal; said main carrier and said securing pieces are shaped into compatible trough and blocks, or are disposed with through holes and then integrated by inserting a pin.

Therefore, the present invention has proposed a composite layer intervertebral retrieving device being implantable between two neighboring vertebrae, comprising:

a main carrier having a middle layer as a whole, and a securing groove disposed on respective surfaces thereof;

multiple securing pieces stably coupled to said securing grooves;

characterized in that: said middle layer of the main carrier is a medical macromolecular material having an elastic modulus of between 2 to 22 GPa, which is close to the original elastic modulus of the vertebrae of between 12 to 18 GPa; said securing pieces are fabricated from materials that are affinitive to and easily fused with osteocytes, and are capable of being imaged by medical penetrating light sources;

a surface of each of said securing pieces is securely coupled to said securing groove, while another surface is disposed with multiple securing bulges; when the device is implanted between the vertebrae, the securing pieces of the device come into direct contact with the osteocytes of the neighboring vertebrae, with the securing bulges pressing against said osteocytes, so as to allow osteoblasts of the vertebrae to grow and fuse with the device; said middle layer has an elastic modulus close to that of the vertebrae to prevent damage to the vertebrae from the stress shielding effect.

The main carrier of said composite layer intervertebral retrieving device has securing grooves disposed on two opposing surfaces, and said securing pieces are coupled to said corresponding securing grooves. Moreover, said main carrier may also be disposed with securing grooves on all surfaces thereof, such that the six surfaces of the upper, lower, front, rear, left and right directions are disposed with securing grooves to be coupled to said securing pieces, so as to allow the device to come into full contact with the vertebrae without being restricted in any directions, and induce the osteocytes of the vertebrae to grow and become fused with said securing pieces.

Said main carrier of the composite layer intervertebral retrieving device is fabricated from a medical polyetheretherketone material, such as PEEK and polyetherketoneketone (PEKK), which has an elastic modulus close to that of the vertebral bone, and this prevents the vertebrae from being further damaged by the stress shielding effect.

Said securing pieces of the composite layer intervertebral retrieving device is fabricated from a medical titanium alloy, a cobalt-chrome-molybdenum alloy, or tantalum metal, so as to induce the osteocytes of the vertebrae to grow and become fused therewith.

In the composite layer intervertebral retrieving device, each of said securing grooves of the main carrier has a recess disposed thereon, and each of said securing pieces is correspondingly disposed with a protruding block to engage with the recess, so as to allow the securing pieces to be securely coupled and fixed on said securing grooves of the main carrier.

The composite layer intervertebral retrieving device further includes multiple pins; as the securing pieces and the middle layer of the main carrier are disposed with multiple through holes, the pins are securely inserted into the through holes of a securing piece, the middle layer of the main carrier, and another securing piece after the main carrier has been coupled to the securing pieces.

The composite layer intervertebral retrieving device may also be implanted into a space enclosed by the vertebral cortical bone.

The composite layer intervertebral retrieving device can be imaged by medical light sources that include a medical X-ray system, a CT scanner, a medical ultrasound scanner, a MRI scanner, or any adequate medical scanning equipment.

For the composite layer intervertebral retrieving device, the directions of upper, lower, front, and rear refer to that of a patient lying face-down on the operation table. The upper end of the lying patient's spine, or his/her head is the upper direction, whereas the lower end of the lying patient's spine, or his/her feet is the lower direction. The direction where the operator sees forward is the frontal direction, and the opposite is the rear direction. In addition, the top and bottom are used to refer to the specified directions, either at the highest or the lowest point in relation to each other, and are indicated by diagrams and numbers.

### BRIEF DESCRIPTION OF DRAWINGS

FIGS. 1a, 1b, 1c, and 1d are four schematic views showing a composite layer intervertebral retrieving device according to four preferred embodiments of the present invention.

FIGS. 2a and 2b are disassembled views showing a conical form of the composite layer intervertebral retrieving device. (FIG. 2a is the preferred representation).

FIGS. 3a and 3b are schematic views showing a cylindrical form of the composite layer intervertebral retrieving device being implanted between an upper and a lower vertebrae.

FIG. 4 is a schematic view showing a bent form of the composite layer intervertebral retrieving device being implanted into a spinal chamber enclosed by the cortical bone of a vertebra to provide support.

### DETAILED DESCRIPTION OF PREFERRED EMBODIMENTS

The present invention can be better understood by referring to the preferred embodiments and accompanying diagrams thereof, in which:

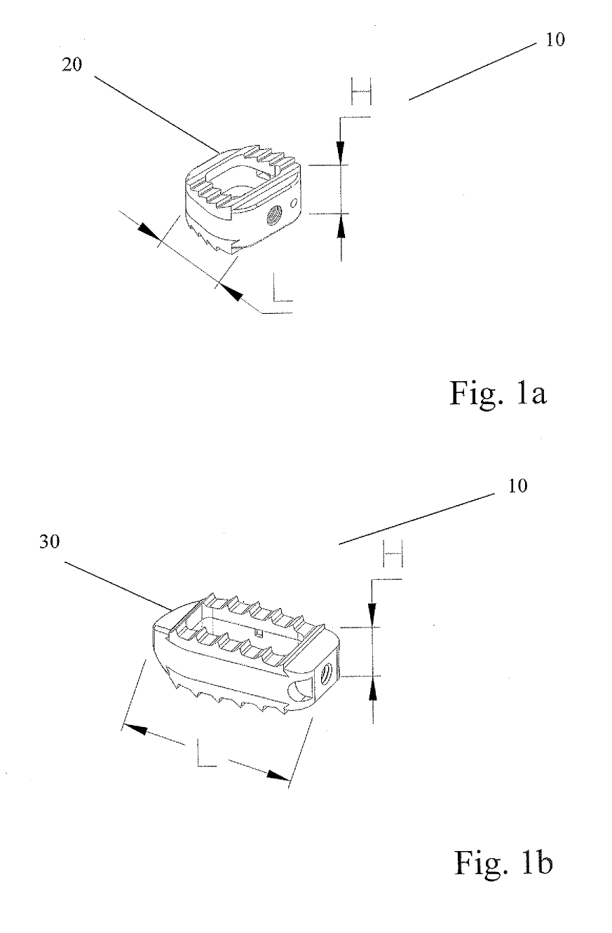

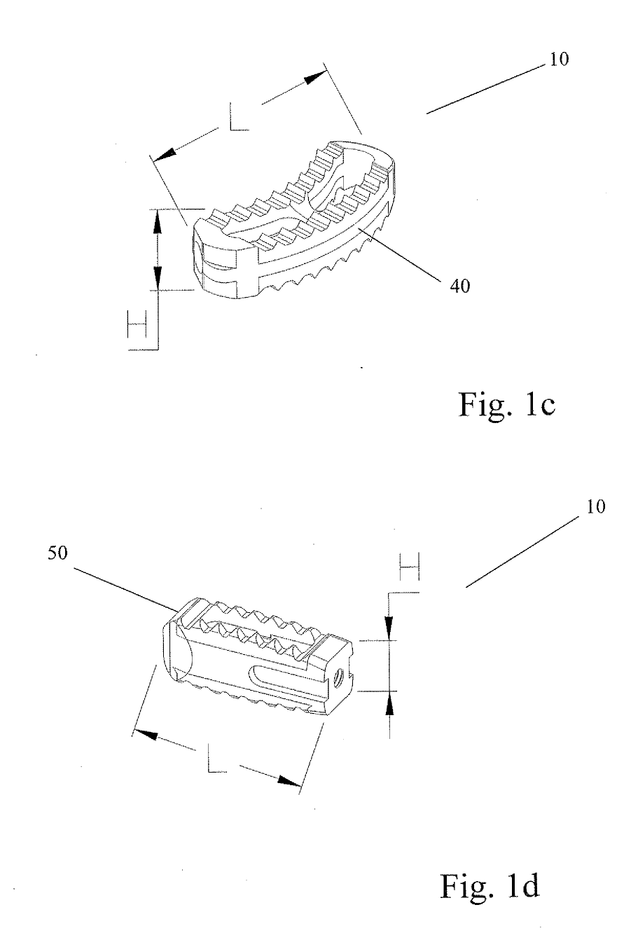

FIGS. 1a, 1b, 1c, and 1d are four representations of a composite layer intervertebral retrieving device 10 of the present invention, which includes a rectangular form 20, a conical form 30, a bent form 40, and a cylindrical form 50; all of which are implantable between two neighboring vertebrae, such as between two cervical vertebrae or lumbar vertebrae.

The preferred embodiments of the present invention employ the conical form 30, the bent form 40, and the cylindrical form 50 as examples.

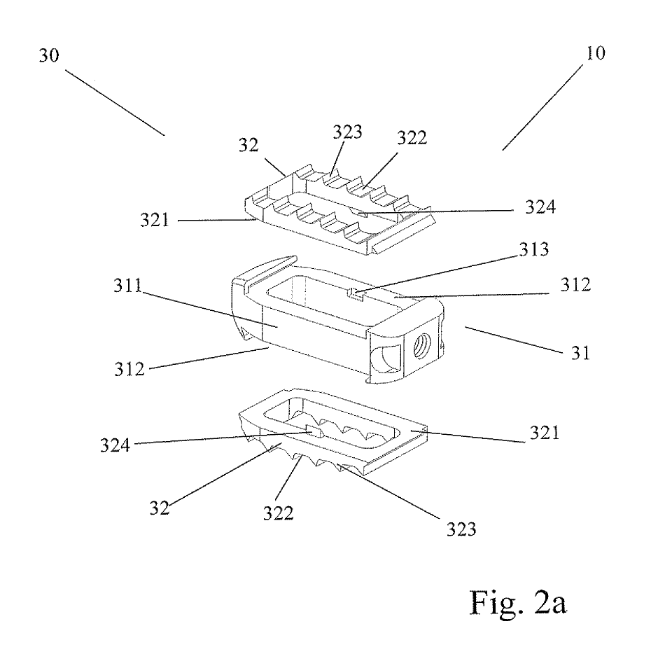

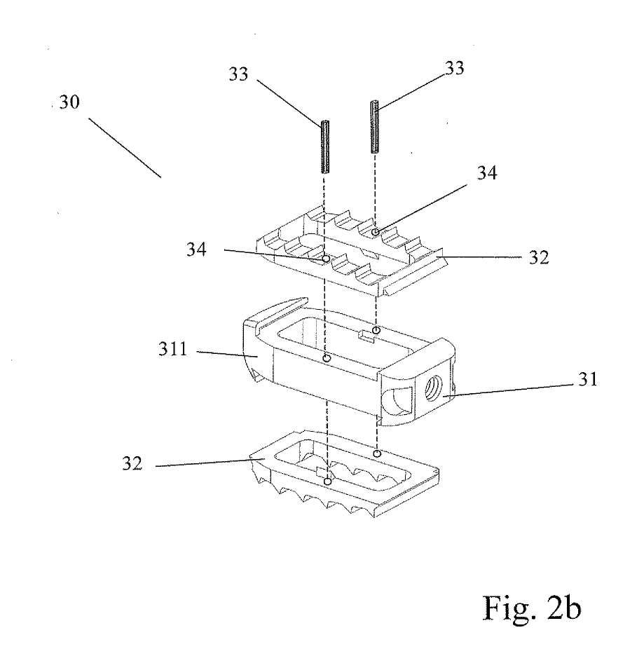

In FIGS. 2a and 2b, said conical form 30 of the composite layer intervertebral retrieving device comprises:

a main carrier 31 having a middle layer 311 as a whole, and securing grooves 312 disposed on surrounding surfaces thereof, and the main carrier has securing grooves disposed on upper and lower surfaces thereof in this embodiment;

multiple (two) securing pieces 32 stably coupled to said upper and lower securing grooves 312 of the main carrier;

and the device is characterized in that: said middle layer 311 of the main carrier is a medical macromolecular material having an elastic modulus of between 2 to 22 GPa, which is close to the original elastic modulus of the vertebrae of between 12 to 18 GPa;

said securing pieces 32 are fabricated from materials that are affinitive to and easily fused with osteocytes, and are capable of being imaged by medical light sources;

a surface 321 of each of said securing pieces is securely coupled to said securing groove 312, while another surface 322 is disposed with multiple securing bulges 323; when the conical form 30 of the composite layer intervertebral retrieving device is implanted between the vertebrae, the securing pieces 32 come into direct contact with the osteocytes of the neighboring vertebrae, with the securing bulges 323 pressing against said osteocytes, so as to allow osteoblasts of the vertebrae to grow and fuse therewith.

Said middle layer 311 of the main carrier has an elastic modulus similar to that of the vertebrae, so as to prevent damage to the vertebrae from the stress shielding effect.

In FIG. 2a, the main carrier 31 has securing grooves 312 disposed on two opposing upper and lower surfaces, and said securing pieces 32 are coupled to the corresponding securing grooves 312.

Furthermore, said main carrier 31 is fabricated from a medical polyetheretherketone material, such as PEEK and PEKK, which has an elastic modulus similar to that of the vertebral bone, and this prevents the vertebrae from being further damaged by the stress shielding effect. Said securing pieces 32 are fabricated from a medical titanium alloy, a cobalt-chrome-molybdenum alloy, or tantalum metal, so as to induce the osteocytes of the vertebrae to grow and become fused therewith.

Said securing grooves 312 of the main carrier has a recess 313 disposed thereon, and each of said securing pieces 32 is correspondingly disposed with a protruding block 324, so as to allow the securing pieces to be securely coupled and fixed on said securing grooves 312 of the main carrier.

As FIG. 2b shows, said conical form 30 of the composite layer intervertebral retrieving device may further include multiple (two) pins 33; as the securing pieces 32 and the middle layer 311 of the main carrier are disposed with multiple (two) through holes 34, the pins 33 are securely inserted into the through holes 34 of a securing piece 32, the middle layer 311 of the main carrier, and another securing piece 32 after the main carrier 31 has been coupled to the securing pieces 32, so that said main carrier 31 and said securing pieces 32 are fixed together more securely.

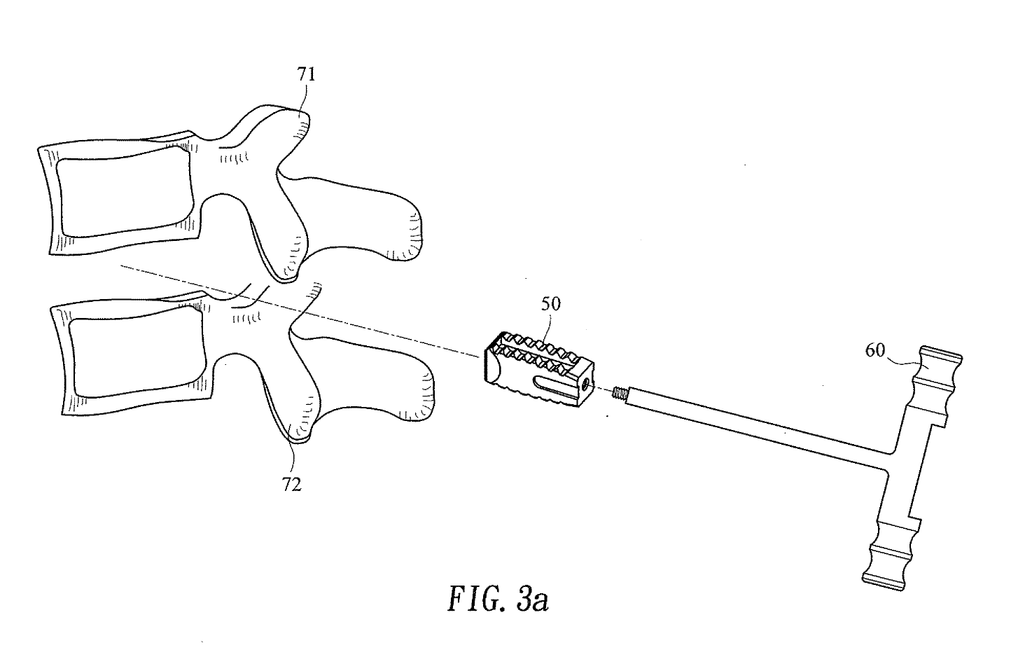

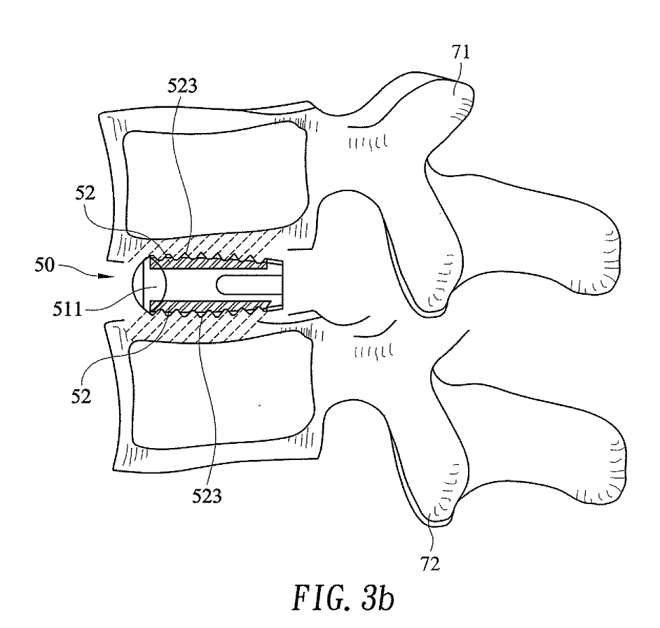

FIGS. 3a and 3b show said cylindrical form 50 of the composite layer intervertebral retrieving device being implanted between an upper vertebra 71 and a lower vertebra 72 by using an implantation tool 60.

Said middle layer 511 of the main carrier from the cylindrical form 50 is fabricated from a medical macromolecular material having an elastic modulus of between 2 to 22 GPa, such as PEEK and PEKK, and is close to the original elastic modulus of the upper and the lower vertebrae 71, 72, which are between 12 to 18 GPa. Thus the elastic modulus of the middle layer 511 of the main carrier is similar to that of the vertebrae 71 and 72, thereby preventing the vertebrae 71 and 72 from being damaged by the stress shielding effect.

Said securing pieces 52 are fabricated from materials that are affinitive to and easily fused with osteocytes, and are capable of being imaged by medical light sources, such that with the presence of said securing pieces 52, said device can be made visible by medical light sources that includes a medical X-ray system, a CT scanner, a medical ultrasound scanner, a MRI scanner, or any adequate medical scanning equipment; when said cylindrical form 50 is implanted between the vertebrae 71 and 72, said securing pieces 52 come into direct contact with the osteocytes of the neighboring vertebrae 71 and 72, with the securing bulges 523 pressing against said osteocytes, so as to allow osteoblasts of the vertebrae to grow and fuse therewith and result in stable integration therebetween.

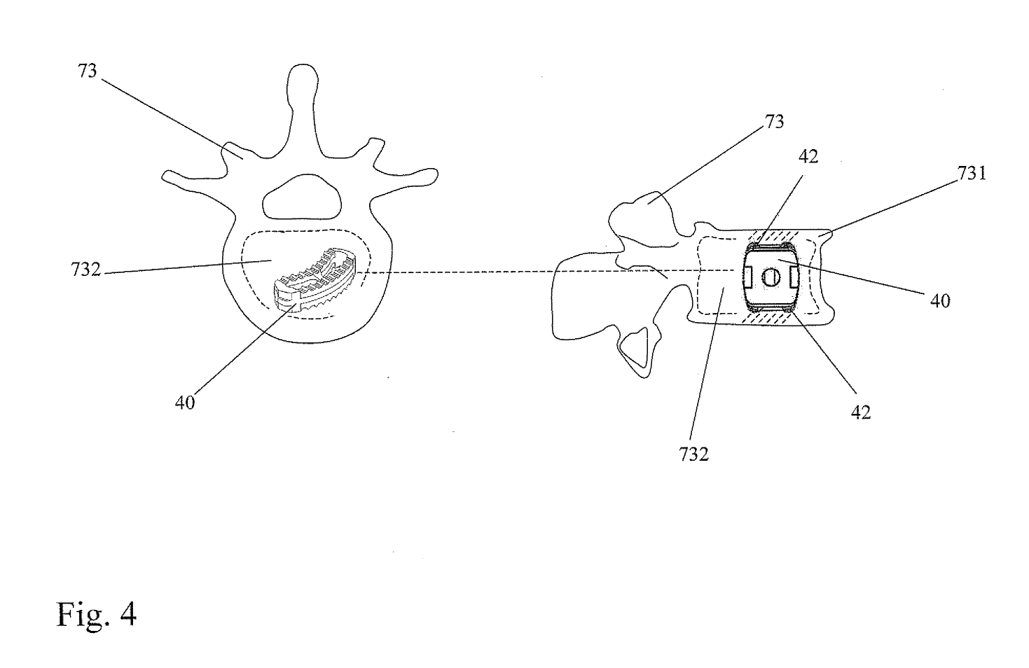

FIG. 4 shows that said bent form 40 of the composite layer intervertebral retrieving device may also be implanted into a spinal chamber 732 enclosed by the cortical bone 731 of a vertebra 73, in order to support the vertebra 73 and prevent fracturing and collapsing, or the securing pieces 42 are allowed to directly touch the osteocytes of the vertebrae 73, so as to allow osteoblasts of the vertebrae to grow and fuse therewith, thereby stabilizing the vertebrae.

What is claimed is:

1. A composite layer intervertebral retrieving device, being implantable between two neighboring vertebrae, comprising:
a main carrier having a middle layer as a whole, and a securing groove provided on surfaces thereof;
multiple securing pieces stably coupled to said securing grooves;
characterized in that: said middle layer of the main carrier is a medical macromolecular material having an elastic modulus of between 2 to 22 GPa, which is close to the original elastic modulus of the vertebrae of between 12 to 18 GPa; and said securing pieces are fabricated from a material that is affinitive to and easily fused with osteocytes, and is capable of being imaged by medical penetrating light sources;
a surface of each of said securing pieces is securely coupled to said securing groove, while another surface is disposed with multiple securing bulges; when the device is implanted between the vertebrae, the securing pieces of the device come into direct contact with the osteocytes of the neighboring vertebrae, with the securing bulges pressing against said osteocytes, so as to allow osteoblasts of the vertebrae to grow and fuse therewith; said middle layer has an elastic modulus similar to that of the vertebrae to prevent damage to the vertebrae from the stress shielding effect.

2. The device of claim 1, wherein said main carrier has securing grooves disposed on two opposing surfaces, and said securing pieces are coupled to said corresponding securing grooves.

3. The device of claim 1, wherein said main carrier is fabricated from a medical polyetheretherketone material.

4. The device of claim 1, wherein said securing pieces are fabricated from a medical titanium alloy, a cobalt-chrome-molybdenum alloy, or tantalum metal.

5. The device of claim 1, wherein each of said securing grooves of the main carrier has a recess disposed thereon, and each of said securing pieces is correspondingly disposed with a protruding block to engage with said recess, so as to allow the securing pieces to be securely coupled and fixed on said securing grooves of the main carrier.

6. The device of claim 1, wherein the device further comprises multiple pins, and the securing pieces and the main carrier are provided with multiple through holes; after the main carrier has been coupled to the securing pieces, the pins are securely inserted into the through holes of a securing piece, the middle layer of the main carrier, and another securing piece.

7. The device of claim 1, wherein the device can be implanted into a space enclosed by the cortical bone of a vertebra.
